(19) United States
(12) Patent Application Publication
(10) Pub. No.: US 2005/0222055 A1
(43) Pub. Date: Oct. 6, 2005

(54) Heterodimeric conjugates of neomycin-oxazolidinone, their preparation and their use

(76) Inventors: Jaehoon Yu, Sungdong-ku (KR); Jongkook Lee, Kangseo-ku (JP); Miyun Kwon, Seocho-ku (KR); Ae Nim Pae, Songpa-ku (KR); Hun Yeong Koh, Kangbuk-ku (KR)

Correspondence Address: NATH & ASSOCIATES, 1030 15th STREET, NW, 6TH FLOOR, WASHINGTON, DC, 20005, US

(21) Appl. No.: 10/502,539

(22) PCT Filed: Jul. 4, 2002
(86) PCT No.: PCT/KR02/01268

§ 371 Date: Jul. 27, 2004

(30) Foreign Application Priority Data
Feb. 8, 2002 (KR) 2002-7495

(51) Int. Cl. C07H 15/20; A61K 31/704

(52) U.S. Cl. 514/39; 536/13.2

(57) ABSTRACT

The present invention relates to novel oxazolidinone derivatives represented as following compound I and a process for the preparation thereof. The compounds of the present invention have wide antibacterial spectrums superior antibacterial activity and low toxicity. Therefore, it can be expected to use as novel antibacterial agent. 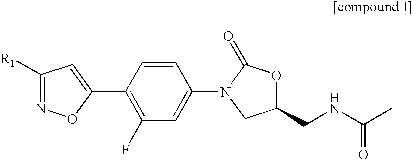
wherein, R₁ is alkylcarboxyl group or —CH₂R₂ (wherein, R₂ is OH, argido group, —OR₃ (wherein, R₃ is C₁₋₄ alkyl, methansulfonyl, p-toluensulfonyl, carboxyl, C₁₋₄ alkylcarboxyl, C₁₋₄ alkylcarbonyl, benzyloxycarbonyl, or imidazolylcarbonyl), or —NHR₄).

### TECHNICAL FIELD

The present invention relates to heterodimeric conjugates of neomycin-oxazolidinone of formula 1, their preparation and their use as an antiviral agent or an antibacterial agent.

### BACKGROUND OF THE INVENTION

Currently most drugs use protein, the final product of genes, as target molecules, which take up 70-80% of the total drugs. However, as RNAs, which are encoding proteins, were found to be pharmaceutical target molecules, intensive and extensive attention has been paid to anti-sense drugs capable of interacting with RNAs.

Research into the morphology of RNA target molecules revealed that the RNA target molecules must be self-base paired to form the most stable form. RNAs have characteristic two- and three-dimensional structures resulting from self-base pairing. In an RNA molecule, bases are paired with other intramolecular bases to create a stem, while a stretch of non-paired bases forms an internal loop. The characteristic three-dimensional stem-loop structure is base sequence-specific, forming a stable pocket to which small molecules can bind well.

The pocket-like RNA structure to which active research has been recently directed can be easily found in ribosomes, which are in vivo protein factories. According to recent reports, it has been disclosed that 20 residues in the decoding region A site of 16S rRNA are highly conserved and are targeted by aminoglycoside, and RNA-binding compound. For example, aminoglycoside with amino groups is known to be the target of binding at the 16S A site, a rRNA of a specific site negatively charged, being the drug generally used showing positive electric charge at physiological pH. According to the NMR structure research, it has been reported that the structure of the stem forms an extended loop which is widened a little by having the aminoglycoside bound at the stem of the RNA. However, the aminoglycoside bound with the RNA as above has a disadvantage in its specificity. That is, even though the aminoglycoside positively charged binds well to the binding site negatively charged, such binding is not specific. As a matter of fact, aminoglycoside has a binding force of about microM to any RNAs with a two-dimensional or three-dimensional structure. Due to not having a specific binding force, the pharmaceutical efficiency of aminoglycoside decreases.

Also, extensive and various attempts have been made to make compounds of low specificity highly specific. Aminoglycosides with no specific binding properties have been made to bind to specific RNAs in various ways. For example, first, there were suggested homodimers of aminoglycosides, with the aim of improving the binding to specific RNAs. Since an associated form of two identical sites with a certain binding force is generally known to show a more potent binding force, homodimers of aminoglycosides are expected to be more specific for some RNAs. However, a significant change is observed in the binding force of a homodimer of aminoglycoside only when the RNA has two or more binding sites for the aminoglycoside, with no observation of a significant change in specificity for the compound. Alternatively, heterodimers are developed, in which aminoglycoside is associated with different kinds of compounds with new functional groups. Tor and his colleagues of Scripps Research Institute reported that a heterodimer in which acridine, a small compound, is associated with aminoglycoside, is about 100-fold more specific to the RRE RNA motif, compared to an individual aminoglycoside. Acridine plays an important role in increasing the binding force in general by recognizing both the base of the bulge projected at the stem self-base paired and the acridine. Likewise, heterodimers associate two molecules which can recognize two difference sites.

According to prior art, the RNA binding site of aminoglycoside is the stem of the RNA, which indicates that aminoglycoside is shape specific to stems, but not base sequence specific. Therefore, compounds bound to RNA motifs with specific sequences form a heterodimer wherein a compound which recognizes the specific structure of the loop, and the aminoglycoside combined specifically to stems are associated. The association of these two compounds for preparing the said compound is very important in enhancing specificity. The present inventors selected chloramphenicol among the compounds known to bind well and tried to bind it with neomycin, a compound showing the highest binding force among aminoglycosides. The association of these two compounds synthesized the two sites the least effective among the pharmaceutical efficiency of neomycin and chloramphenicol, and the thus synthesized heterodimers show a highly enhanced specificity at several RNA motifs.

In the meanwhile, the exact site of one of the recently developed antibiotics, the oxazolidinone compound has not been found out yet, but the compound displays a possibility to bind to sites other than binding sites such as chloramphenicol or microride which were known to bind to 23S rRNAs as an RNA binding material showing pharmaceutical efficiency by binding to the 23S rRNA.

Leading to the present invention, the intensive and thorough research into RNA-specific drugs, conducted by the present inventors with the aim of solving problems encountered in prior arts, resulted in finding that neomycin-oxazolidinone heterodimers, in which neomycin is linked through a spacer to oxazolidinone, can more strongly bind to specific RNAs and recognize both the stems and loops of the RNA molecules, with base sequence specificity.

### DISCLOSURE OF THE INVENTION

The present invention relates to neomycin-oxazolidinone heterodimer represented by the following formula 1. 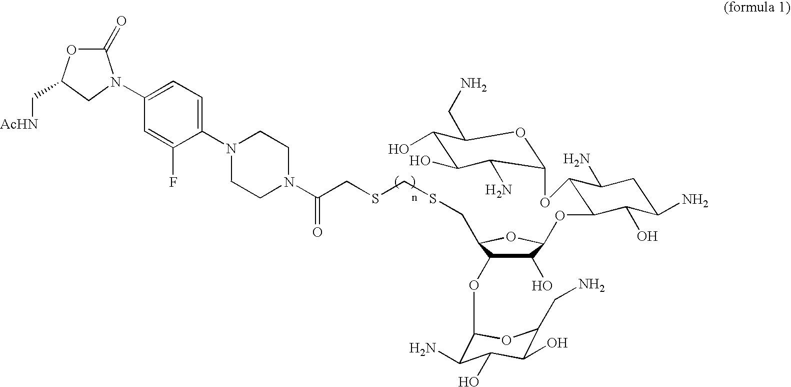
wherein,
n is an integer of 2-10, preferably 6,
Ac is acetyl group.

As represented by the above formula 1, the neomycin-oxazolidinone heterodimer of the present invention has a structure which connects the main structure of neomycine and oxazolidinone with a spacer having carbon chains of a suitable length.

Particularly, among oxazolidinone and aminoglycoside, known to show a strong binding force to RNA loops, neomycin showing the strongest binding force to RNAs was bound using a site the least affected by the pharmaceutical effect of the two compounds as a spacer. The spacer comprises dimercapto compounds, preferably the carbon number of the spacer is 6.

The neomycin-oxazolidinone heterodimer of the present invention can recognize both stems and loops at the same time, and thus, enhance the binding force to specific RNAs (16S rRNA, 23S rRNA). In accordance with the following embodiments, neomycin-oxazolidinone heterodimers are specific to 16S rRNA or 23S rRNA showing a strong binding force. Specifically, the binding force between neomycin-oxazolidinone heterodimers and 16S rRNA or 23S rRNA is more than 60 times and 30 times enhanced compared with neomycin, and is more than 300 times and 4000 times enhanced compared with oxazolidinone. In addition, RRE RNA shows a result wherein the change of binding force is even lower than that of neomycin monomers. This indicates that the binding force at RNAs which is caused by heterodimers differs in how much they increase according to its species. Further, even though the RNA motif has both stems and loops, it turns out that only the rRNAs with specific base sequences show a specific binding force. 23S rRNA showed an increase in the binding force wiht neomycin-oxazolidinone heterodimers even though it has a very short RNA sequence. This indicates that the binding force of heterodimer at a relatively long RRE RNA is decreased compared with that of a neomycin monomer, and thus, the heterodimer of this present invention binds to specific RNAs.

The present invention comprises a method for preparing neomycin-oxazolidinone heterodimer represented by the following reaction scheme 1, which particularly comprises steps of;
Reacting the compound of formula 2 with the compound of formula 3 in the presence of base to obtain the compound of formula 4 (step 1); and
Reacting the obtained compound of formula 4 with a deprotective agent to prepare the compound of formula 1 (step 2). 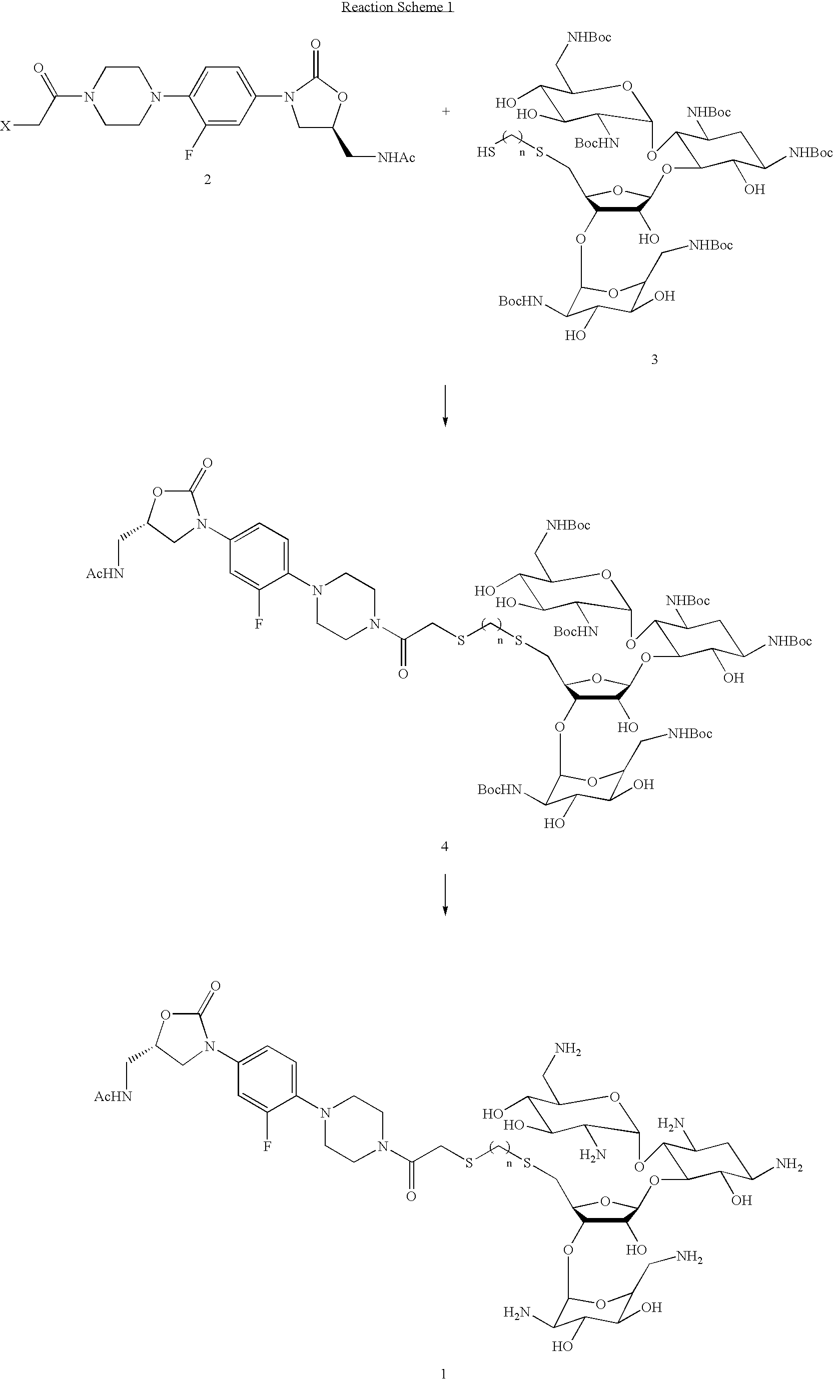
wherein, n is an integer of 2-10, preferably 6,
X is a F, Cl or Br,
Ac is acetyl group,
Boc is t-butyloxycarbonyl group.

In step 1, the compound of formula 2 is reacted with the compound of formula 3 in the presence of base at room temperature for 5-10 hours to obtain the compound of formula 4. The base is K₂CO₃, Na₂CO₃ or Cs₂CO₃, preferably Cs₂CO₃. At this time, a used solvent is dimethylforamide, dimethyl sulfoxide or acetonitrile, preferably dimethylforamide.

In step 2, the deprotective agent, to deprotect t-butyloxycarbonyl of formula 4, is hydrochloric acid, hydrofluoric acid, sulfuric acid, nitric acid, acetic acid or trifluoroacetic acid, preferably trifluoroacetic acid. Also, as presented by the following reaction scheme 2, the compound of formula 5 is reacted with the compound of formula 6 in the presence of base to obtain the compound of formula 2. Preferably, the base is pyridine, and the used solvent is CH₂Cl₂. Also, the reaction temperature is preferably 0° C., and the reaction time is 2 hours. 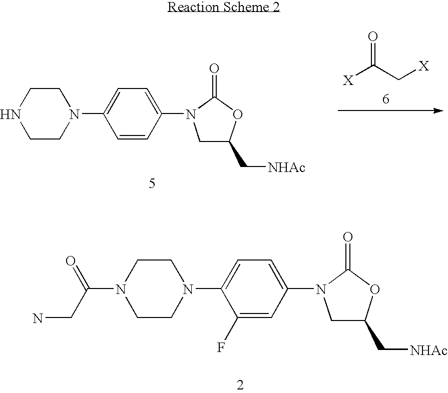
wherein,
Ac is acetyl group,
X is independently Cl, Br or F.

As presented by the following reaction scheme 3, the compound of formula 7 is reacted with dimercapto compound in the presence of base to obtain the compound of formula 3. 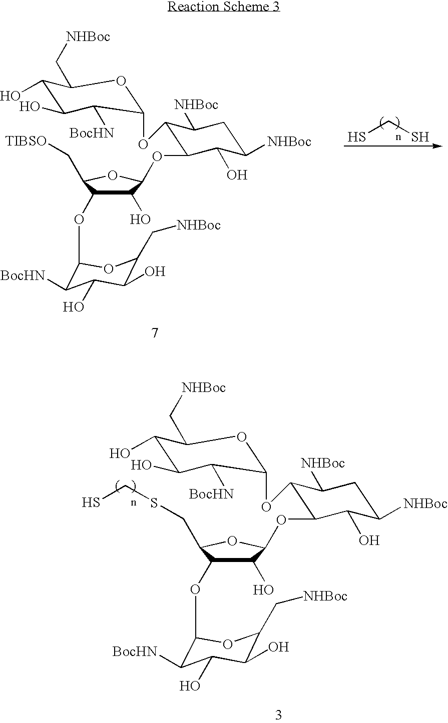
wherein,
n is an integer of 2-10, preferably 6,
Boc is t-butyloxycarbonyl group,
TIBSO is triisopropylsulfonyl group.

The compound of formula 7 is prepared from neomycine by a conventional method.

Dimercapto compound is reacted with the compound of formula 7 in the presence of base to obtain the compound of formula 3. Preferably, the dimercapto compound is 1,6-hexanditiol, the base is K₂CO₃, Na₂CO₃ or Cs₂CO₃, and the solvent is DMF, DMSO or acetonitrile.

Also, the present invention comprises an antiviral agent and an antibacterial agent having heterodimeic conjugates of neomycin-oxazolidinone as an active ingredient.

The neomycin-oxazolidinone heterodimers of the present invention recognize both stems and loops as RNA motif to show a strong bonding force to specific RNAs (16S rRNA, RRE RNA, 23S rRNA) present at ribosomes of the pathogenic organism, whereby enables it to be effectively used as an antiviral agent or an antibacterial which can inhibit the synthesis of protein of a pathogenic organism.

That is, the heterodimeric conjugates of neomycin-oxazolidinone can be formulated into various dosage forms for oral or parenteral administration. For formulation, pharmaceutically acceptable diluents, expedients and/or carriers may be used, including fillers, thickeners, binders, wetting agents, disintegrants, surfactants, etc. Solid dosage forms for oral administration are exemplified by tablets, pills, powders, granules, and capsules. These solid forms are prepared by admixing neomycine-oxazolidinone heterodimer of formula 1 with at least one expedient, such as starch, calcium carbonate, sucrose, lactose, gelatine, etc. In addition to expedients, lubricants such as magnesium styrate may be added.

Liquid dosage forms for oral administration exemplified by suspensions, internal solutions, emulsions, syrups, etc., may comprise simple diluents, such as water and liquid paraffin, as well as wetting agents, sweeteners, aromatics, and/or perspectives.

Dosage forms for parenteral administration include sterile aqueous solutions, non-aqueous solvents, suspensions, emulsions, freeze-dried agents, suppositories, etc. For formulation of non-aqueous solvents and suspensions, vegetable oils, such as propylene glycol and polyethylene glycol, olive oil or injectable esters such as ethyl oleate, may be used. As basee for syppositories, witepsol, macrogol, Tween 61, cocoa oil, laurinic acid, and glycerogelatine are useful.

The amount of the active ingredient actually administered ought to be determined in light of various relevant factors including the absorptance of active components in vivo, the water active values, the rate of excretion, the age, sex and body of the individual subject, and the severity of the subject's symptoms. In genernal, the compound of neomycin-oxazolidinone heterodimer may be administrated in a total dose of 0.1-50 mg per 1 kg a day to adults in 1 or various administrations, preferably, 0.1-10 mg per 1 kg.

A better understanding of the present invention may be obtained in light of the following examples which are set forth to illustrate, but not construed to limit the present invention.

### EXAMPLE

### Preparation Example 1

### Preparation of the Compound of Formula 2

34 mg (0.10 mmol) of oxazolidinone derivative of formula 5 (Brickner et al., J. Med. Chem., 1996, 39, 673-679) was mixed with 2.0 ml of dichloromethane. Subsequently, 0.025 ml (0.31 mmol) of pyridine and 0.13 ml (0.15 mmol) of bromoacetyl bromide were added to the reaction solution at 0° C. The obtained reaction mixture was stirred for 1 hour and then diluted with acetyl acetate. The obtained mixture was washed with brine to obtain organic layer. The organic layer was dried using anhydrous Na₂SO₄, and concentrated in vacuo. The residue was purified with silicagel chromatography (CH₂Cl₂:MeOH=10:1) to obtain 33 mg of a white solid, the compound of formula 2 (yield 72%).

¹H NMR (CDCl₃, 300 MHz) δ 7.50(dd, J=14.2, 2.5 Hz, 1H), 7.10(dd, J=8.8, 2.4 Hz, 1H), 6.99(t, J=9.0 Hz, 1H), 6.13(t, J=6.1 Hz, 1H), 4.82-4.76(m, 1H), 4.13-3.51(m, 10H), 3.16(t, J=4.9 Hz, 2H), 3.08(t, J=5.0 Hz, 2H), 2.04(s, 3H).

### Example 1

### Preparation of neomycin-oxazolidinone heterodimer

(Step 1): Preparation of the Compound of Formula 4

97 mg (0.072 mmol) of the compound of formula 3 and 33 mg (0.072 mmol) of formula 2 were dissolved in 1.5 ml of DMF, and then 24 mg (0.074 mmol) of Cs₂CO₃ was added to the mixture. The obtained mixture was stirred for 15 hours and the mixture poured into water. The mixture was extracted using 100 ml of EtOAc and the organic layer was washed with brine. The obtained organic layer was dried using anhydrous Na₂SO₄ and concentrated in vacuo. The residue was purified with silicagel chromatography (CH₂Cl₂:MeOH=10:1) to obtain 101 mg of white solid, the compound of formula 4 (yield 81%).

¹H NMR(CD₃OD, 300 MHz) δ 7.53(dd, J=14.5, 2.4 Hz, 1H), 7.18(dd, J=8.8, 2.1 Hz, 1H), 7.07(t, J=9.1 Hz, 1H), 6.68(br d, J=6.68 Hz, 1H), 6.49(br d, J=6.3 Hz, 1H), 5.32(br s, 1H), 5.19(br s, 1H), 4.94(br s, 1H), 4.83-4.75(m, 1H), 4.23-2.60(m, 39H), 1.97(s, 3H), 1.65-1.24(m, 64H).

(Step 2): Preparation of Heterodimeric Conjugates of neomycin-oxazolidinone

101 mg (0.059 mmol) of the compound of formula 4 was mixed with 1.5 ml of trifluoroacetic acid (TFA) and then stirred at room temperature for 30 minutes. The obtained mixture was concentrated in vacuo, and then purified with prep-HPLC (prep-C18 column, H₂O containing 0.1% TFA: MeCN containing 0.1 TFA=70:30) and lyophilized to obtain 78 mg of white solid, the compound of formula 1 (yield 69%).

¹H NMR(D₂O, 300 MHz) δ 7.09(dd, J=14.0, 1.9 Hz, 1H), 6.91-6.87(m, 2H), 5.70(d, J=4.0 Hz, 1H), 5.04(d, J=2.0 Hz, 1H), 4.94(d, J=1.4 Hz, 1H), 4.04-2.69(m, 38H), 2.45(dd, J=13.4, 7.6 Hz, 1H), 2.27(q, J=7.3 Hz, 4H), 2.17-2.11(m, 1H), 1.62(s, 3H), 1.54(q, J=12.6 Hz, 1H), 1.29-1.18(m, 4H), 1.05-1.01(m, 4H);

¹³C NMR(D₂O, 75 MHz) δ 175.2, 171.2, 163.9(q, J=35.2 Hz), 157.0, 134.7, 120.9, 116.7(q, J=29.1 Hz), 116.2, 110.8, 109.0, 108.7, 96.0, 95.5, 85.8, 80.5, 78.9, 75.4, 74.1, 73.2, 72.8, 71.3, 70.4, 69.8, 68.3, 68.0, 67.7, 54.0, 51.4, 51.2, 51.0, 50.0, 48.7, 48.6, 46.2, 42.1, 40.8, 40.7, 34.6, 33.0, 32.1, 31.9, 29.1, 28.7, 28.3, 27.9, 27.7, 22.1.

### Preparation Example 2

### Experimental Preparation of Specific RNAs

A sense DNA of 16S rRNA (sequence listing 1) and an antisense DNA of 16S rRNA (sequence listing 2); a sense DNA of RRE RNA (sequence listing 3) and an antisense DNA of RRE RNA (sequence listing 4); and a sense DNA of 23S RNA (sequence listing 5) and an antisense DNA of 23S RNA (sequence listing 6) was prepared according to the present invention.

Paricularly, the two DNAs comprising sense and antisense (2.5 nanomole, respectively), 5× buffer solution (200 mM Tris-HCl, 30 mM MgCl₂, 10 mM spermidine, 50 mM NaCl, pH 7.9; 20 μl), 100 mM DL-dithiotheitol (DTT; 20 μl), four nucleotide tri phosphate mixture (2.5 mM, 20 μl), T7 RNA polymerase (50 units/mL; 1 μl) and diluted water (34 μl) were mixed and cultured at 37° C. for 2 hours. And then 1 unit/ml of Rnase-free RQ1 Dnase was added to the mixture and cultured at 37° C. for 10 minutes. 100 μl of a PCI mixture solution (phenol:chloroform:isopropanol=25:24:1) was added to the obtained mixture, mixed at room temperature for 5 minutes and then centrifuged at 1400 rpm for 10 minutes. The obtained upper solution was poured into a new tube and the RNA was concentrated using the ethanol precipitation method. The obtained RNA was purified by performing electrphoresis at 6% of polyacrylamide containing 7.0 M urea at a current of 20 mA for 30 minutes. After cutting the RNA band lightened with a UV flashlight and transferring it to a new tube, 500 μl of elution buffer (0.5 M ammonium acetate, 1 mM EDTA, 0.2% SDS, pH 8.0) was added to the mixture and it was left alone at 37° C. for 4 hours. The liquated RNA was transferred to a new tube and purified by phenol extraction and ethanol precipitation. The amount of RNA purified can be certified using a 260 nm UV spectrum.

### Experimental Example 1

### Determination of Bond Constant Between the Compound of the Present Invention and Specific RNAs

The paromomycin bonded with tetramethylrhodamine (hereinafter referred to as “CPR”) is used as a luminescence fluorescent probe. The luminescence anisotropy is measured by establishing a thermostat of 20° C. at the Perkin-Elmer LS-50B luminescence spectroscope. The luminescence absorptance of CRP is 510 nm and its luminescence fluorescent is observed at 550 nm. At least 7 measurements were made to obtain one data, wherein the maximum value and the minimum value were excluded and the average of the other 5 measurements was used as the data. The luminescence was measured at an elution buffer using 140 mM NaCl, 5 mM KCl. 1 mM MgCl₂, and 20 mM HEPES pH 7.5. The equation measuring the bond constant (Kd) between CRP and the prepared RNA is represented by the following equation 1:
A=A₀+DNA{([RNA]₀+[CRP]₀+K_{d})−([RNA]₀+[CRP]₀+K_{d})²−4[RNA]₀[CRP]₀^{1/2}}/2
wherein,
A is the luminescence anisotropy value of CRP when RNA is present,
A₀ is the luminescence anisotropy value of CRP when RNA is not present,
DA is the difference of luminescence anisotropy value between various RNA concentrations when RNA is not present,
[RNA]₀ is the initial concentration of RNA,
[CRP]₀ is the initial concentration of CRP,
K_{d} is the bond constant.

If the newly prepared compound is added to the solution after inducing the bonding of the said RNA and CRP, CRP is separated from RNA by a competitive bonding reaction and the compound to be measured achieves a KD value by bonding with RNA. The equation to achieve the KD value is represented by the following equation 2, and K_{d} and KD are achieved using the non-linear curve fitting method not a linear fitting method. The results are shown in the following table 1.
[Aminoglycoside]₀{KD(A_{∞}−A₀)/[K_{d}(A_{√}−A₀)+1]}×{[RNA]₀−K_{d}(A−A₀)/(A_{∞}−A₀)−[CRP]₀(A−A₀)/(A_{∞}−A₀)} (Equation 2)
wherein,
KD is the bond constant between RNA and the new aminoglycoside,
[Aminoglycoside]₀ is the initial concentration of aminoglycoside to be measured,
A is the luminescence anisotropy value when the bond is being measured,
A_{∞} is the luminescence anisotropy value when the bonding is completed,

A₀ is the luminescence anisotropy value when everything is free.

TABLE 1
Comparison of the binding force of heterodimers against
each RNA (microM)

| | | | Neomycin- |
|---|---|---|---|
| | Neomycin | oxazolidinone | oxazolidinone |
| 16s rRNA | >2 | 10.3 | 0.034 |
| RRE RNA | 0.18 | Non bonding | 0.54 |
| 32S RNA | >2 | 260 | 0.063 |

As shown in the above table 1, since neomycin-oxazolidinone heterodimers show an enhanced binding force with 16S rRNA or 23S rRNA compared with neomycin, an increase in the binding force of general heterodimers was observed in this present invention. However, as for RRE RNAs, the change of the binding force decreased compared with that of neomycin itself, which indicates that the change of the binding force of heterodimers depends on the RNA being observed and its [symbol] [symbol] . Specifically, the binding force between neomycin-oxazolidinone heterodimers and 16S rRNA or 23S rRNA is more than 60 times and 30 times enhanced compared with neomycin, and is more than 300 times and 4000 times enhanced compared with oxazolidinone. The above results indicate that even though the 3 RNA motifs prepared in accordance with the present invention have both stems and loops, only the rRNA with base specific sequences showed an increase in the binding force to neomycin-oxazolidinone heterodimers, and such increase was the highest for 23S rRNAs. 23S rRNA showed an increase in the binding force to neomycin-oxazolidinone heterodimers even though it has a very short RNA sequence. This indicates that the binding force of heterodimer at a relatively long RRE RNA shows a decreased binding force compared with that of a neomycin monomer, and thus, the heterodimer of this present invention binds to specific RNAs.

### Experimental Example 2

### Acute Toxicity Experiment on Parenteral Administration of Rats

In order to find out whether the compound of formula 1 has acute toxicity, the following experiment was performed.

A six week old specific pathogen-free (SPF) SD rat was used in the acute toxicity experiment. The neomycin-oxazolidinone heterodimer of the present invention was suspended in 1 ml of physiological saline and administered into the muscles of two rats in the amount of 1 mg/kg. Then, the present inventors observed the life and death of the animal, clinical symptoms, weight variance, and performed haematological examination and blood-biochemical examination. Further, they observed with the naked eye whether there were any changes at the abdominal organ and thorasic organ after performing necropsy.

As a result, none of the animals administered with the experimental material showed any specific clinical symptoms or death. Further, toxicity change was not observed in weight variance, haematological examination, blood-biochemical examination, necropsy observations and diagnosis, either. From the above results, the compounds used in this experiment are evaluated to be safe substances, since they do not cause any toxic change in rats up to the level of 10 mg/kg, and the oral administration minimum lethal dose (LD₅₀) is much higher than 10 mg/kg.

### INDUSTRIAL APPLICABILITY

As disclosed above, the neomycin-oxazolidinone heterodimers of the present invention show a stronger binding force with 16S rRNA, RRE RNA and 23S RNA compared with neomycin or oxazolidinone, recognizes both stems and loops of the RNA motif, and also has a specific bond with base sequences comprising RNA. Therefore, the increase of specificity in recognizing RNAs not only enhances the pharmaceutical efficacy of the drug but also enables the neomycin-oxazolidinone heterodimers to be effectively used as an antiviral agent or an antibacterial due to the reduced side effect which can be caused by non-specific drugs.

1. A neomycin-oxazolidinone heterodimer represented by formula 1: 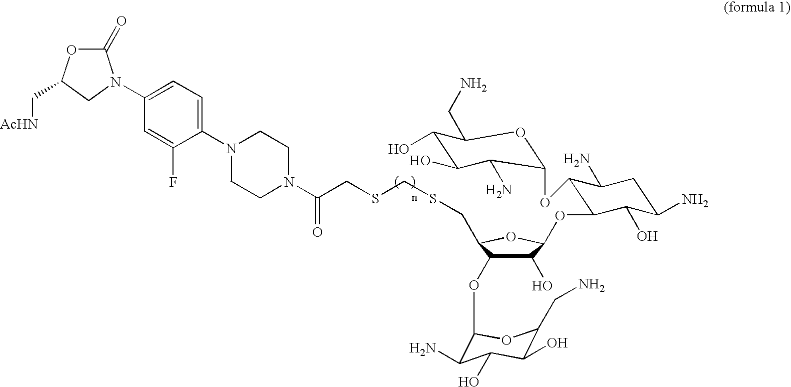
wherein, n is an integer of 2-10,
Ac is acetyle group.

2. The neomycin-oxazolidinone heterodimer according to claim 1, wherein n is 6.

3. The neomycin-oxazolidinone heterodimer according to claim 1, wherein the neomycin-oxazolidinone heterodimer forms a specific bond with 16S rRNA, RRE RNA or 23S RNA.

4. The neomycin-oxazolidinone heterodimer according to claim 3, wherein the specific bond recognizes both stems and loops of the RNA motif.

5. The neomycin-oxazolidinone heterodimer according to claim 3, wherein the specific bond is a base sequence-specific bond comprising RNA.

6. A method for preparing neomycin-oxazolidinone of claim 1, comprising steps of:
reacting the compound of formula 2 with the compound of formula 3 in the presence of base to obtain the compound of formula 4 (step 1), and
reacting the obtained compound of formula 4 with a deprotective agent to prepare the neomycin-oxazolidinone heterodimer of claim 1 (step 2): 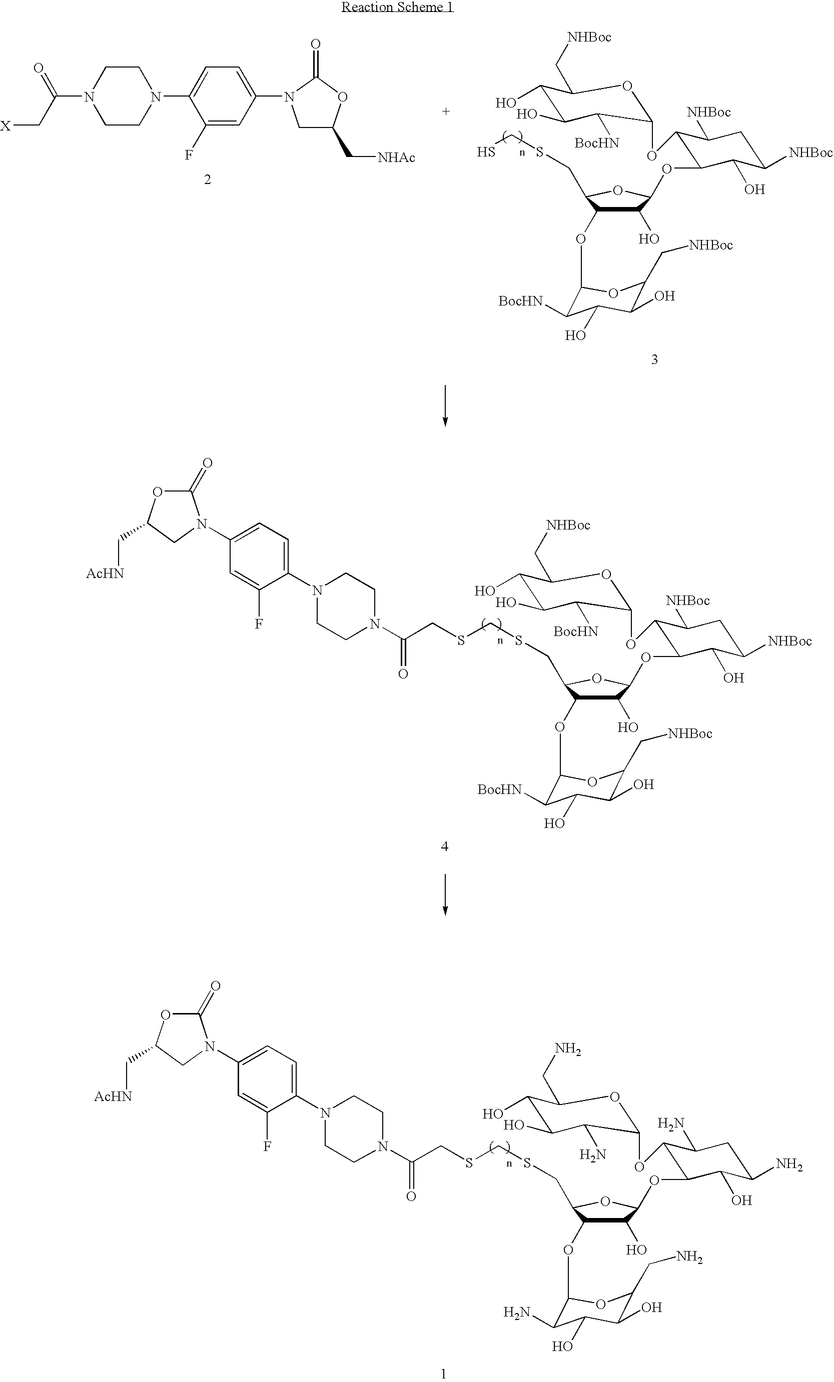
wherein, n is an integer of 2-10,
X is F, Cl or Br,
Boc is t-butyloxycarbonyl group.

7. The method according to claim 6, comprising steps of reacting the compound of formula 5 with the compound of formula 6 in the presence of pyridine to obtain the compound of formula 2: 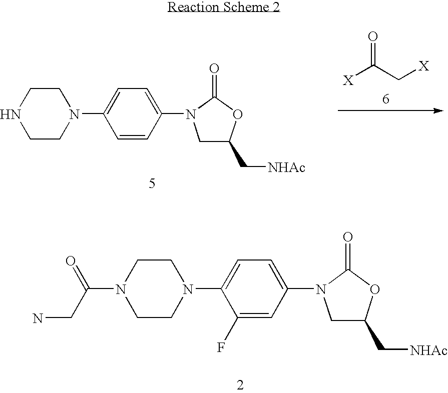
wherein, Ac is acetyl group,
X is independently Cl, Br or F.

8. The method according to claim 6, wherein the base of step 1 is K₂CO₃, Na₂CO₃ or Cs₂CO₃ and the deprotective agent is hydrochloric acid, sulfuric acid, nitric acid, acetic acid or trifluoroacetic acid.

9. An antiviral agent or an antibacterial containing neomycin-oxazolidinone heterodimer of claim 1 as an active ingredient.


SEQUENCE LISTING (6 sequences)

SEQ ID NO: 1 - DNA, 49 bp, Artificial Sequence
  16s sense
aatttaatacgactcactatagggcgtcacaccttcgggtgaagtggcc

SEQ ID NO: 2 - DNA, 49 bp, Artificial Sequence
  16s antisense
ggccacttcacccgaaggtgtgacgccctatagtgagtcgtattaaatt

SEQ ID NO: 3 - DNA, 52 bp, Artificial Sequence
  RRE RNA sense
aatttaatacgactcactatagggtgggcgcagcttcggctgacggtacacc

SEQ ID NO: 4 - DNA, 52 bp, Artificial Sequence
  RRE RNA antisense
ggtgtaccgtcagccgaagctgcgcccaccctatagtgagtcgtattaaatt

SEQ ID NO: 5 - DNA, 25 bp, Artificial Sequence
  23S rRNA sense
ggactcgctgtgaagatgcagtgta

SEQ ID NO: 6 - DNA, 25 bp, Artificial Sequence
  23S rRNA antisense
tacactgcatcttcacagcgagtcc